(19) United States
(12) Patent Application Publication
(10) Pub. No.: US 2013/0331756 A1
(43) Pub. Date: Dec. 12, 2013

(54) JoJo Vest

(71) Applicant: Joseph Sassone, Metairie, LA (US)

(72) Inventor: Joseph Sassone, Metairie, LA (US)

(21) Appl. No.: 13/491,542

(22) Filed: Jun. 7, 2012

(51) Int. Cl. A61F 5/00

(52) U.S. Cl. 602/19

(57) ABSTRACT

The JoJo Vest is a patient aid to be worn by a person. It has no moving parts and is made of 100% pre-washed cotton, with three adjustable plastic buckles/fasteners down the front that guarantee a proper fit.

This vest is used to help caregivers, nurses, and physical therapists have more support, mobility and flexibility in helping patients move in and out of beds, chairs and/or wheelchairs. The vest also helps in stabilizing patients who need support in walking with the help of the caregiver.

### ABSTRACT

The JoJo Vest is a patient aid to be worn by a person. It has no moving parts and is made of 100% pre-washed cotton, with three adjustable plastic buckles/fasteners down the front that guarantee a proper fit.

This vest is used to help caregivers, nurses, physical therapists have more support, mobility and flexibility in helping patients move in and out of beds, chairs and/or wheelchairs. The vest also helps in stabilizing patients that need support in walking with the help of the caregiver.

### BACKGROUND OF THE INVENTION

1. The Field of Endeavor/Invention

The present invention relates to the medical field, i.e., in rehabilitation/physical therapy, giving caregivers an advantage in enhancing patient's mobility.

2. Related Art

The problem it addresses is that current belts used in medical facilities do not always give sufficient stability and support to move patients. These belts tend to put more strain on the caregiver or therapist to keep the patient mobile and stable when moving them from one location to another such as a bathroom or walking with them during the patient's rehabilitation and/or physical therapy.

### BRIEF SUMMARY OF THE INVENTION

The JoJo Vest is made for the medical and physical rehabilitation industry.

The JoJo Vest uses the same mechanics a person would use to put on a regular vest. The vest is made specifically for immobile patients that need assistance in standing and walking.

### LIST OF FIGURES FOR DRAWINGS

### Front View

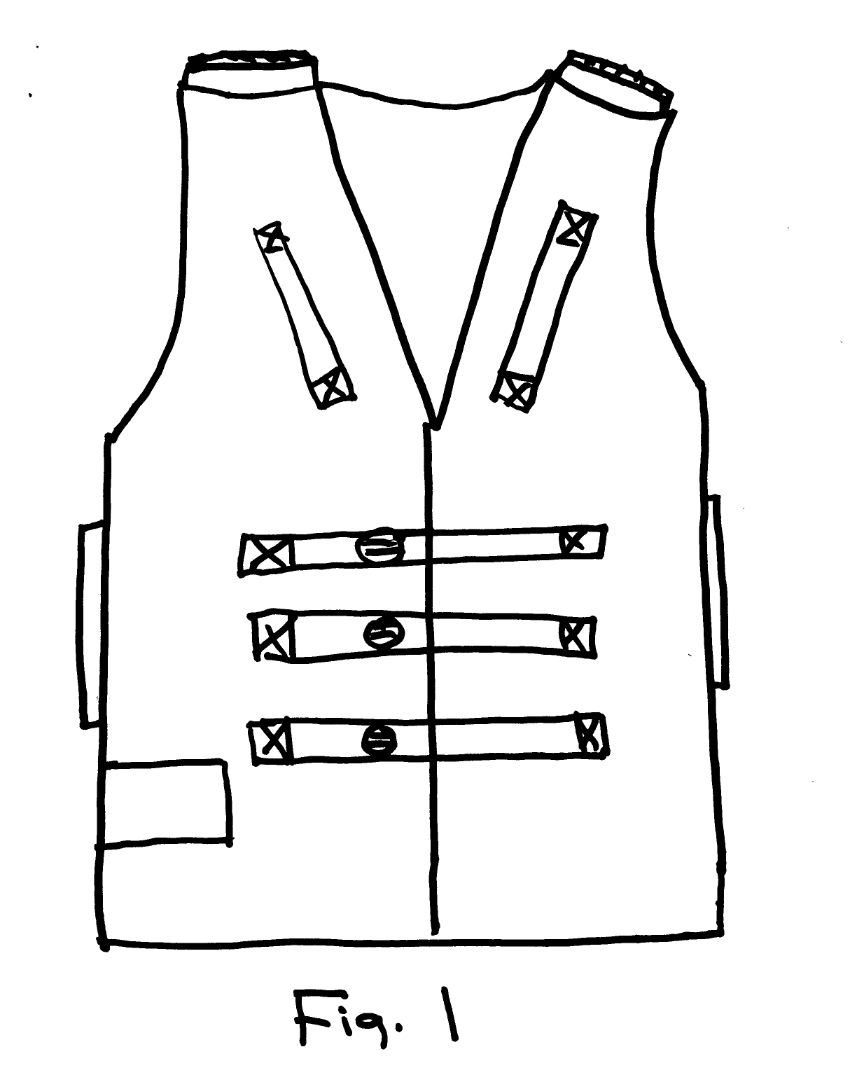

FIG. 1—Adjustable Straps and Plastic Locking Clips

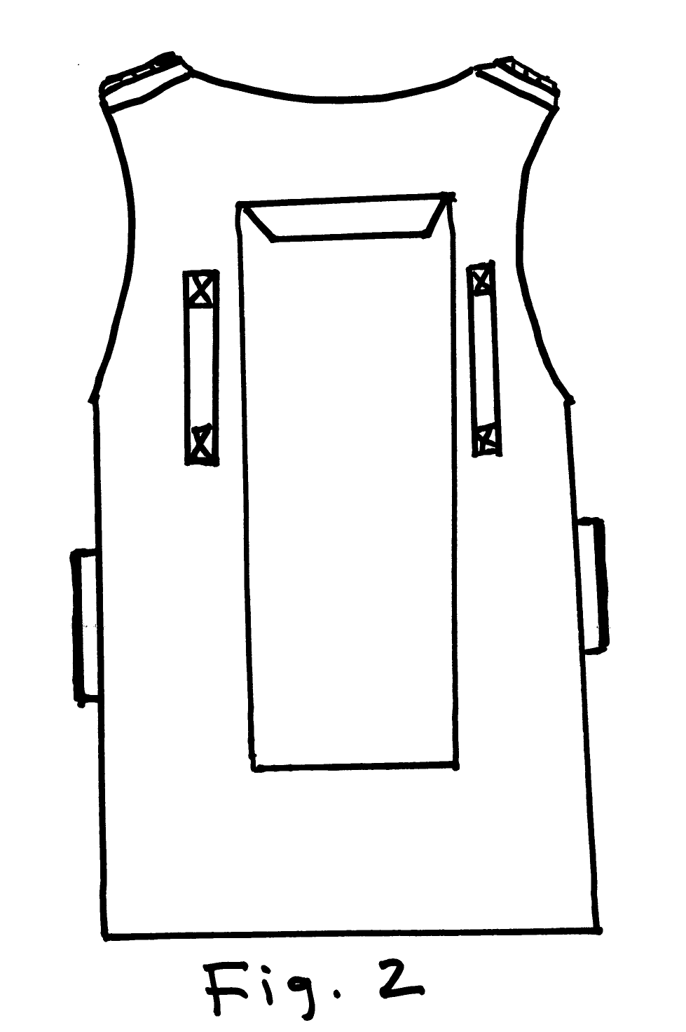

FIG. 2—Side Handles

FIG. 3—Front Handles

FIG. 4—Shoulder Handles

FIG. 5—Clear Plastic Patient I.D. Window

FIG. 6—100% Cotton Pre-washed denim fabric

### Back View

FIG. 7—Back Handles

FIG. 8—Rear Pouch for Plastic Insert (Approx. ⅛″ thick×7 ¾″ W×18″ L)

### DESCRIPTION OF INVENTION

The JoJo Vest is put on a patient by the caregiver or physical therapist. One arm goes through an armhole, and then the other arm goes through the other armhole. Once the vest is on the person, there are three (3) clips that fasten in front to ensure the closing of the vest. The clips are adjustable by pulling the belt material through the clip to ensure a proper fit. (FIG. 1) After the proper fit has been established by the caregiver, they can assist the patient with standing and maintain control of the patient, by securely grabbing the strategically fastened handles on the front, sides, back and shoulders on the vest. (FIGS. 2,3,4,7) The patient can be on the edge of the bed, in a chair, wheelchair or in any position that would require assistance for the patient to stand. There is also a pouch located in the center back of the vest for a high impact plastic panel to be inserted to ensure the patient's back is supported and straight when being assisted. (FIG. 8) When a patient is ready to return to a sitting position, the caregiver grabs the handles and lowers the patient slowly and safely, maintaining control through out the process. (FIGS. 2,3,4,7) The vest is removed by unfastening the three (3) front clips (FIG. 1) and removing the vest with the same mechanics a person would use in removing any vest or shirt.

JoJo Vest is made from pre-washed, 100% cotton, is machine washable and can be reused multiple times. The vest comes in 6 different sizes. The JoJo Vest is unique, new idea and product that will by all intensive purposes become the standard device in helping to maintain stability and control of recovering patients, elderly and feeble patients, and patients in rehab and physical therapy, in achieving more mobility in standing and walking, with the assistance of their caregiver.

1. A vest comprising of handles, locking clips with adjustable belts, a plastic insert that slides into a padded pouch sewn on the back, for spinal protection and posture adjustment that together provides stability and control in the care of physical needs patients.

2. The vest of claim 1, will be available in various sizes comprising of small, medium, large, extra large and double XX in order to accommodate multiple patients.

3. The vest of claim 1, will be made from a durable 100% pre-washed cotton fabric.

4. The vest of claim 1, can be used multiple times.

5. The vest of claim 1, is a durable product and be cleaned by sliding out the plastic back panel out of its pouch and simply cold wash and line dry.

6. The vest of claim 1, comes with a clear I.D. window stitched to the front bottom right for purpose of verifying patient identity.
